(19) United States
(12) Patent Application Publication
(10) Pub. No.: US 2017/0022123 A1
(43) Pub. Date: Jan. 26, 2017

(54) MANUFACTURE OF ETHYLENE FROM ETHANOL

(71) Applicant: John E. Stauffer, Greenwich, CT (US)

(72) Inventor: John E. Stauffer, Greenwich, CT (US)

(21) Appl. No.: 14/803,601

(22) Filed: Jul. 20, 2015

(51) Int. Cl. C07C 1/24

(52) U.S. Cl. CPC C07C 1/24; C07C 2521/12

(57) ABSTRACT

Ethylene is produced from ethanol in a one-step process by reacting ethanol with hydrogen chloride over a silica alumina catalyst.

### FIELD OF THE INVENTION

The present invention relates to a process for manufacturing ethylene from ethanol. The process is conducted in one step by reacting ethanol with hydrogen chloride over a solid catalyst, thereby dehydrating the ethanol to render ethylene and water.

### BACKGROUND

There is growing interest in the manufacture of ethylene from renewable sources. This trend is motivated by concerns about global warming and the uncertainty about prices of petroleum feedstock. As a result, leading manufacturers of ethylene are turning to ethanol as a raw material. The ethanol may be supplied by the fermentation of sugar from either sugar cane or corn syrup. The chemistry for producing ethylene from ethanol is well known. It is straightforward in concept, having been the subject of much academic research. Ethylene is formed by the dehydration of ethanol in the vapor phase reaction when the alcohol is passed over a catalyst of gamma aluminum oxide at a temperature in the range of 348° to 428° C.

The commercial application of this technology, however, presents certain problems. The catalyst life is limited. Byproducts are formed, including such impurities as heavy residues, as well as light ends. Furthermore, an inherent disadvantage of the chemistry is the coproduction of ether, which must be recovered and recycled in the process.

For these and other reasons, there is an incentive to develop new technology for the production of ethylene from ethanol. Thus, it is a goal of the present invention to provide a process that is efficient, robust and versatile in its use. These and other advantages, features, and characteristics of the process of the present invention will become apparent from the following description and the figure that is included.

### BRIEF DESCRIPTION OF THE DRAWING

FIG. 1 is a block diagram showing the principal features of the process. Intermediate and product streams are indicated on the flow sheet.

### DETAILED DESCRIPTION

Two chemical reactions can be identified in the process of the present invention. These reactions take place simultaneously and occur in intimate contact. These two reactions are shown by the following equations:

C₂H₅OH+HCl→C₂H₅Cl+H₂O 1

C₂H₅Cl→C₂H₄+HCl 2.

The net result of these reactions is as follows:

C₂H₅OH→C₂H₄+H₂O 2.

As shown above, the ethyl chloride produced in the first reaction is consumed in the second reaction. Similarly, the requirement of hydrogen chloride for the first reaction is supplied by the second reaction. Depending on the physical design of the reactor and the feed streams, the quantity of hydrogen chloride can be minimal.

The conditions for the process of the present invention are all-important. A catalyst or several catalysts are needed to promote the reactions of equations 1 and 2. Fortunately, one catalyst will suffice for both reactions, a condition that makes possible the key feature of the present invention, namely, a one-step process.

Several catalysts for the process have been investigated, but one catalyst stands out as being superior. It provides the best all-around performance. That catalyst is silica alumina.

The literature discusses the chemistry shown by equation 1. Various alkyl chlorides can be made from the corresponding alcohols making use of hydrogen chloride as a direct chlorinating agent in organic synthesis. Thus, methyl, ethyl, and propyl chlorides can be made by a vapor phase reaction of the given alcohol. The reaction is carried out at a temperature of 200° to 300° C. over an alumina silica catalyst mass containing 0.01 to 1 percent Na₂O.

Supplementing these results, experimental data has been obtained for the reaction given by equation 2. While the effective catalysts for this reaction are numerous, three catalysts in particular stand out. These substances are activated charcoal, zinc chloride, and silica alumina. Activated charcoal shows some activity, but it is less effective than other catalysts. Zinc chloride has the disadvantage of its volatility so that steps need to be taken to maintain its activity.

Using silica alumina as a cracking catalyst, the reaction as shown by equation 2 functions best at a temperature in the range of 325° to 375° C. This reaction is endothermic so that heat must be supplied to the reactor. Under these conditions, the results are near perfect.

Combining the chemistry for the chlorination of ethanol with the science for cracking ethyl chloride, a one-step operation can be obtained. In this unified process, the catalyst of choice is silica alumina, it being active for both chlorination and cracking. While this catalyst is effective over a wide spectrum of temperatures, the range can be narrowed to 300° to 325° C. without sacrificing efficiency. The reaction is carried out at a pressure in the range of 1 to 10 atmospheres.

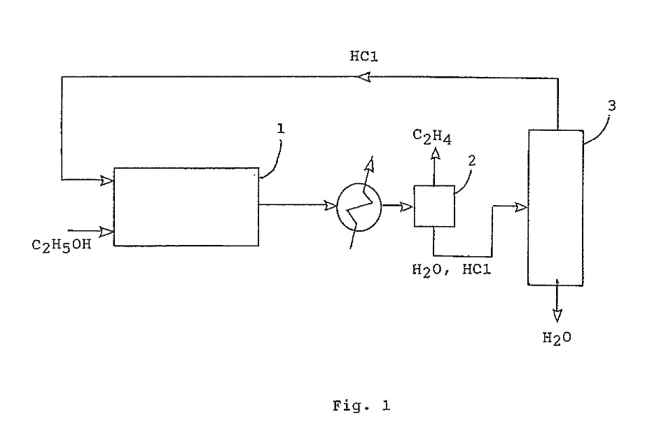

The advantages of the present invention are best illustrated by referring to FIG. 1. Ethanol and hydrogen chloride are fed to reactor 1, which typically may consist of a shell and tube design. The effluent is cooled in a heat exchanger and then passed to phase separator 2 where the ethylene product is recovered. Hydrochloric acid is sent to distillation column 3 to recover hydrogen chloride for recycle to reactor 1.

### EXAMPLE

In a laboratory experiment, ethyl chloride was cracked over a commercial silica alumina catalyst at 350° C. to give a near quantitative yield of ethylene. The catalyst had a composition of 12.4 weight percent Al₂O₃ and 87.3 weight percent SiO₂ and it had a surface area of 300 m² per gm. The pellet density was 0.99 kg per liter and porosity equaled 57 volume percent. No loss of catalyst activity was noted during the experiment.

### SUMMARY

A process is provided for the synthesis of ethylene from ethanol in a one-step process. In the process ethanol vapor and hydrogen chloride are passed over a solid catalyst to dehydrate the ethanol to form ethylene.

In the overall reaction, hydrogen chloride reacts with ethanol to form water and ethyl chloride. The latter in turn is cracked to form ethylene and release hydrogen chloride. In effect, the hydrogen chloride acts simply as a catalyst, passing through the process unchanged.

Purified product is produced by separating water and hydrogen chloride from the ethylene. The hydrogen chloride is recycled in order to provide a self-contained process.

1-3. (canceled)

4. A process for the manufacture of ethylene from ethanol comprising the steps of:
reacting ethanol with hydrogen chloride over a solid catalyst chosen from the group consisting of activated charcoal and zinc chloride in a temperate range of about 325° C. to 375° C. to dehydrate the ethanol and render ethylene and water; and
separating the ethylene from the water.

5. The process defined in claim 4 carried out at a pressure of between 1 and 10 atmospheres.

6. A process as defined in claim 4 wherein the reactions
C₂H₅OH+HCl→C₂H₅Cl+H₂O
C₂H₅Cl→C₂H₄+HCl
are carried out simultaneously to yield
C₂H₅OH→C₂H₄+H₂O.
